(19) United States
(12) Patent Application Publication
(10) Pub. No.: US 2026/0224325 A1
(43) Pub. Date: Aug. 6, 2026

(54) RADIO-OPAQUE MARKER AND SYSTEM FOR TUMOUROUS TISSUE

(71) Applicant: VAL-CHUM, LIMITED PARTNERSHIP, Quebec (CA)

(72) Inventors: Moishe LIBERMAN, Westmount (CA); Rodin CHERMAT, Montreal (CA); Mark VARTAZARMIAN, Montreal (CA); Alexandre ABID, Montreal (CA); Saman NAGHIEH, Pointe-Claire (CA)

(21) Appl. No.: 19/147,417

(22) PCT Filed: Jan. 10, 2024
(86) PCT No.: PCT/CA2024/050022

§ 371 Date: Jul. 11, 2025

Related U.S. Application Data
(60) Provisional application No. 63/438,365, filed on Jan. 11, 2023.

(51) Int. Cl. A61B 90/00; A61B 10/02

(52) U.S. Cl. CPC A61B 90/39; A61B 10/0233; A61B 2090/3908; A61B 2090/3966; A61B 2090/3987; A61B 2090/3991

(57) ABSTRACT

A marker may have an elongated body including a radio-opaque material. A penetrating tip tapers at a leading end of the elongated body. One or more anchoring member define a catching surface facing toward a trailing end of the elongated body, the catching surface forming a protrusion relative a downstream portion of the elongated body. The marker may be part of an assembly with a guide releasably attached to the trailing end of the marker. A method for delivering a radio-opaque marker in soft tissue may also be provided.

### CROSS-REFERENCE TO RELATED APPLICATION

The present application claims the priority of U.S. Patent Application No. 63/438,365, filed on Jan. 11, 2023 and incorporated herein in its entirety by reference.

### TECHNICAL FIELD

The application relates radio-opaque markers (a.k.a., radiopaque) of the type used for locating tissue landmarks in medical imaging.

### BACKGROUND

Radio-opaque markers, also known as fiducial markers, are small metal pieces (typically gold) that may be as small as the size of a grain of rice, in the shape of spheres, cylinders or coils, placed in contact with or in a tumor in order to precisely determine its position to deliver the maximum dose of radiotherapy, to facilitate its resection, etc. Fiducial markers are typically used in lesions located in the soft tissues of the chest (chest wall, lung), abdomen (liver, gallbladder, kidneys, pancreas), pelvis (prostate), or head and neck. The implantation of a fiducial marker is an image-guided procedure that may be performed by an interventional radiologist, for example, in preparation for specific radiotherapy techniques, such as stereotactic radiosurgery (SRS), stereotactic body radiotherapy (SBRT), or protontherapy. Fiducial markers can also be used to facilitate image-guided resection, especially for small lesions that are difficult to access.

In current techniques, the implantation of radio-opaque markers is often performed after biopsy sampling. Accordingly, the implantation of radio-opaque markers results in a repeat of needle sticks, in addition to further steps of locating the tumour for the implantation of the markers at the precise location. Moreover, the separate biopsy and marker implanting results in a multiplication of needles.

### SUMMARY

In one aspect, there is provided a marker comprising: an elongated body including a radio-opaque material, a penetrating tip tapering at a leading end of the elongated body, and at least one anchoring member defining a catching surface facing toward a trailing end of the elongated body, the catching surface forming a protrusion relative a downstream portion of the elongated body.

Further in accordance with the aspect, for instance, the marker has a circular cross-section along its entire length.

Still further in accordance with the aspect, for instance, the penetrating tip has a conical geometry.

Still further in accordance with the aspect, for instance, the at least one anchoring member has a frusto-conical body.

Still further in accordance with the aspect, for instance, the frusto-conical body has a cone angle greater than that of the penetrating tip.

Still further in accordance with the aspect, for instance, the marker has two of the anchoring member.

Still further in accordance with the aspect, for instance, the two anchoring members are separated by a first cylindrical segment of the elongated body.

Still further in accordance with the aspect, for instance, a second cylindrical segment of the elongated body is downstream of a downstream one of the two anchoring members.

Still further in accordance with the aspect, for instance, the first cylindrical segment has a greater diameter than the second cylindrical segment.

Still further in accordance with the aspect, for instance, the catching surface includes a straight radial surface.

Still further in accordance with the aspect, for instance, the marker is a monoblock.

Still further in accordance with the aspect, for instance, the marker includes platinum.

In accordance with another aspect, there is provided an assembly comprising: a marker as described above; and a guide configured to contact a trailing portion of the marker.

Further in accordance with the other aspect, for instance, the guide is a silica guide.

Still further in accordance with the aspect, for instance, the silica guide is a tube.

Still further in accordance with the aspect, for instance, the guide is releasably attached to the trailing portion of the marker.

In accordance with yet another aspect, there is provided a method for delivering a radio-opaque marker in soft tissue, the method comprising: positioning a marker and a guide inside a needle or catheter with the needle or catheter in an area of the soft tissue; with the needle or catheter remaining in the area, pushing the guide with the marker at its leading for the marker to reach the area via the needle or catheter, and pierce through the soft tissue; and pulling the guide out of the needle to separate the guide from the marker.

Further in accordance with the aspect, for instance, the method may include repeating the positioning, the pushing and the pulling while the needle remains in area of the soft tissue.

Still further in accordance with the aspect, for instance, positioning the marker attached to the guide inside the needle or catheter includes positioning the marker attached to the guide inside the biopsy needle.

Still further in accordance with the aspect, for instance, the method may include positioning the needle or catheter in the area of the soft tissue to obtain at least one biopsy sample prior to positioning the marker and the guide in the area of the soft tissue.

### DESCRIPTION OF THE DRAWINGS

Reference is now made to the accompanying figures in which:

FIG. 1 is a schematic view of a radio-opaque marker delivery system and radio-opaque marker in accordance with the present disclosure;

FIG. 2 is a fragmented side view of the radio-opaque marker of FIG. 1;

FIG. 3 is a schematic view of the radio-opaque marker of FIG. 1, with locating guide;

FIG. 4 is a schematic view of the radio-opaque marker delivery system of FIG. 2 in tumourous tissue, prior to insertion of the radio-opaque marker;

FIG. 5 is a schematic view of the radio-opaque marker inserted in the radio-opaque marker delivery system, subsequent to FIG. 4;

FIG. 6 is a schematic view of the radio-opaque marker of FIG. 5 in the tumourous tissue, after removal of the radio-opaque marker delivery system;

FIG. 7 is a radiographic image of a plurality of the radio-opaque markers of the present disclosure in biological tissue;

FIG. 8 is another radiographic image of a plurality of the radio-opaque markers of the present disclosure in biological tissue; and

FIG. 9 is another radiographic image of a plurality of the radio-opaque markers of the present disclosure in biological tissue, with higher radio-contrasting values for the radio-opaque markers.

### DETAILED DESCRIPTION

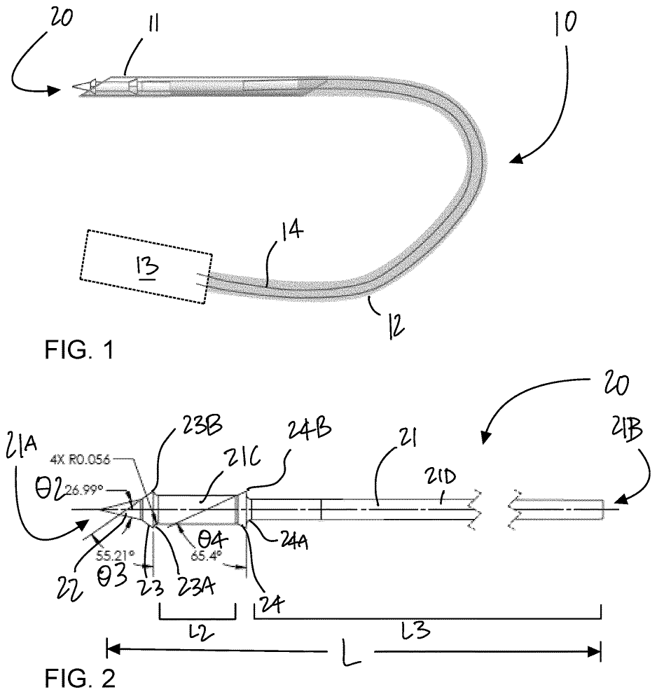

Referring to the drawings and more particularly to FIG. 1, a radio-opaque marker delivery system in accordance with the present disclosure is shown at 10, and is used to deliver a radio-opaque marker or radio-opaque markers 20 in biological tissue, such as tumourous tissue A. In explaining the radio-opaque marker delivery system 10 and the radio-opaque marker 20, reference is made herein to tumour A for simplicity, though the radio-opaque marker delivery system 10 and radio-opaque marker 20 may be used as marker for other anatomical features. The radio-opaque marker 20 may be said to be part of the radio-opaque marker delivery system 10, or may be separate from the radio-opaque marker delivery system 10. The radio-opaque marker 20 may also be known as a radiopaque, fiducial, a fiducial marker, a radiocontrasting marker/fiducial, a radio-contrasting marker/fiducial, etc. The radio-opaque marker delivery system 10 may be used in vivo in a variant, to deliver and locate the radio-opaque marker 20 in the tumour A. The radio-opaque marker 20 may then be detected using appropriate imaging, such as the various forms of radiography (e.g., X-ray, fluoroscopy, etc), ultrasound imaging, computerized tomography, etc. In the case of tumour A, the radio-opaque marker 20 may be used to pinpoint the location of the tumour A for subsequent interventions, such as stereotactic radiotherapy, image-guided surgery, etc.

In FIG. 1, the radio-opaque marker delivery system 10 is shown schematically as having a needle 11 (e.g., piercing needle), and a straight tube, a flexible tube or like hollow wire 12 (e.g., nitinol® hollow wire), and are part of a biopsy sampling system. The illustrated embodiment shows and is described herein as being a biopsy sampling system, but the radio-opaque marker delivery system 10 could be part of other types of medical devices, such as standard needle, syringe, catheter, etc. While the needle 11 and the tube 12 are shown as separate components in FIG. 1, they may be integrated into a single component, such as a single needle or catheter. Moreover, tip shapes other than the one shown in FIG. 1 may be used. Different sizes of needle 11 may be used, but in a variant, the needle 11 is a 22-gauge needle. Biopsy hardware is generally shown as 13 and may include different hardware components, such as a stylet, a syringe, a scope, etc. Examples of such biopsy sampling system or apparatus may include endobronchial ultrasound system (EBUS), endoscopic ultrasound system (EUS), trans-thoracic CT guided system, trans-peritoneal CT guided system, trans-rectal US guided system, CT guided neuro/skeletal system, trans-nasal system, navigation guided system, etc. In a variant, the radio-opaque marker delivery system 10 uses the needle 11 and tube 12 after biopsy tissue sampling has been performed, with the needle 11 remaining on the site of the sampling. The biopsy sampling system may be for concomitant tumor biopsy, e.g., with rapid on-site evaluation (ROSE), prior to insertion of the radio-opaque marker 20.

Still referring to FIG. 1, the radio-opaque marker delivery system 10 may include a locating guide 14, that is configured to deliver the radio-opaque marker 20 through the tube 12 and needle 11, and into the tumour A. The locating guide 14 may take any appropriate form, but may be tubular or may have a female end for being releasably coupled to the radio-opaque marker 20. For example, the locating guide 14 may be a silica tube of medical grade, through other materials may be used.

Referring to FIG. 2, the radio-opaque marker 20 is shown in greater detail. The radio-opaque marker 20 has an elongated body 21. The elongated body 21 of the radio-opaque marker 20 is shown fragmented, as it may extend longer than what is shown in FIG. 2. For example, the radio-opaque marker 20 has a length L extending from 5.354 to 6.354 mm, inclusively, though it may be longer or shorter. The elongated body 21 has a leading end 21A and a trailing end 21B, relative to the direction of delivery. The elongated body 21 may also include cylindrical segments 21C, and 21D. The cylindrical segment 21C may have a length LC of 0.850 to 0.900 mm, inclusively, though it may be longer or shorter. A diameter of the cylindrical segment 21C may be 0.25 to 0.31 mm inclusively, though it may be smaller or greater. The cylindrical segment 21D may have a length of 3.794 to 4.794 mm, inclusively, though it may be longer or shorter. A diameter of the cylindrical segment 21D may be between 0.214 and 0.218 mm, inclusively, though it may be smaller or greater. The end of the cylindrical segment 21D may be beveled, but this is optional. In the illustrated embodiment, a cross-section of the radio-opaque marker 20 along the length L may be circular for the full length L, with a diameter of the cross-section varying depending on surface features defined herein. In other embodiments, the cross-section may not be circular at all, or may have non-circular portions.

The leading end 21A includes a penetrating tip 22. The penetrating tip 22 may have any appropriate shape, but is shown as being a right-circular cone, and forms a taper at the leading end 21A. The cone angle Θ2 may be between 12.5 and 15.0 degrees, inclusively, though it may be more or less. Other shapes could include interrelated fins (e.g., similar to cross-head screwdriver, but with optionally fewer or more fins). An anchoring member 23 may be located downstream of the penetrating tip 22. The anchoring member 23 may have different configurations, but is shown as being a frustoconical formation of a right-circular cone. A diameter of the anchoring member 23 may be between 0.37 and 0.4 mm, inclusively, though it may be smaller or greater. As shown, the anchoring member 23 may have a cone angle Θ3 that may be greater than cone angle Θ2. For example, the cone angle Θ3 may be between 50 and 60 degrees, inclusively, though it may be more or less. In a variant, the cone angle Θ3 of the anchoring member 23 is equal to or greater than cone angle Θ2 of the penetrating tip 21. In a variant, the penetrating tip 22 and the anchoring member 23 form a continuous single cone. The anchoring member 23 could have other shapes, and may for example include one or more fins, as a possibility. The anchoring member 23 projects outwardly from the cylindrical segment 21C and thus forms a catching formation that anchors the radio-opaque marker 20 in place.

The anchoring member 23 then merges with the cylindrical segment 21C. In FIG. 2, the anchoring member 23 merges with a reverse frustoconical formation 23A, but other shapes are possible, including a straight radial face or surface (a.k.a., a radial face that lies in a radial plane, to which a longitudinal axis of the radio-opaque marker 20 is normal), with a smaller frustoconical formation as an example. As observed, a straight radial face portion 23B may be present. The straight radial face portion 23B could for example extend all the way to the cylindrical segment 21C, with or without a fillet, and thus without the reverse frustoconical formation 23A. Either way, the anchoring member 23 defines a step facing in a trailing direction, which step opposes to a withdrawal of the radio-opaque marker 20. This catching formation may be optional.

Another anchoring member 24 may be located downstream of the anchoring member 23, and may be separated from the anchoring member 23 by the cylindrical segment 21C. The anchoring member 24 may have different configurations, but is shown as being a frustoconical formation of a right-circular angle. As shown, the anchoring member 24 may have a cone angle Θ4 that may be smaller than cone angle Θ3. The cone angle Θ4 may be between 60 and 70 degrees, inclusively, though it may be more or less. In a variant, the cone angle Θ3 of the anchoring member 23 is equal to or less than cone angle Θ4 of the anchoring member 24. The anchoring member 24 could have other shapes, and may for example include one or more fins, as a possibility. The anchoring member 24 projects outwardly from the cylindrical segments 21C and 21D, forming another catching formation that anchors the radio-opaque marker 20 in place.

The anchoring member 24 may then merge with the cylindrical segment 21D. In FIG. 2, the anchoring member 24 merges with a reverse frustoconical formation 24A, and a straight radial face portion 24B may be present. Other shapes are possible. The straight radial face portion 24B could for example extend all the way to the cylindrical segment 21D, with or without a fillet, and thus without the reverse frustoconical formation 24A. This catching formation may be optional.

Though two anchoring members are depicted in FIG. 2 for the radio-opaque marker 20, i.e., the anchoring member 23 and the anchoring member 24, there may be a single or single row of anchoring member, or there may be more than two. It may suffice to have single anchoring member to ensure that the radio-opaque marker 20 is retained in the tumour A. To increase the purchase, an additional anchoring member(s) may be present. However, the anchoring members 23 and/or 24 increase the radial footprint of the radio-opaque marker 20, and may thus have an impact on the displacement of the radio-opaque marker 20 in the lumen of the tube 12 (if present), especially of the tube 12 has a curvature as in FIG. 1 of the radio-opaque marker delivery system 10. In a variant, if the radio-opaque marker 20 is to be used in a curved tube 12, the length L is selected to be inferior to a circular arc segment corresponding to a maximum curvature in the tube 12 (e.g., 22 gauge), enabling the radio-opaque marker 20 to travel through the tube 12.

Thus, in a variant, the anchoring members 23 and 24 (if both present) each define a catching surface(s) facing toward the trailing end 21B of the elongated body 21. The geometry of the anchoring members 23 and 24 (if both present) tapers in a toward a leading end of the marker 20, so as to facilitate penetration of the marker 20 in soft tissue. On the other hand, the catching surface of the anchoring members 23 and 24 (shown as 23A, 23B, 24A, 24B) form a protrusion (e.g., a step, a steep surface) relative a downstream portion (e.g., cylindrical segments 21C, 21D) of the elongated body 21. The elasticity of the soft tissue will collapse toward the segments 21C and 21D, and the members 23 and 24 will be caught in the soft tissue. The narrowing of the elongated body 21, with steepness, immediately downstream of the anchoring members 23 and 24 enables the soft tissue to capture the marker 20.

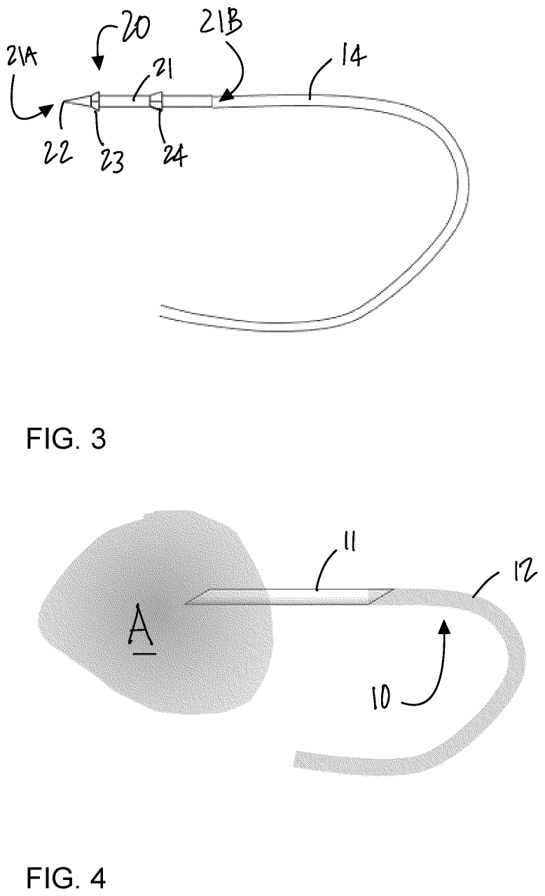

Referring to FIG. 3, the radio-opaque marker 20 is shown having the locating guide 14 at its trailing end 21B. The smaller diameter of the cylindrical segment 21D may enable same to be fitted inside the locating guide 14. The material of the locating guide 14 is selected for the assembly of the locating guide 14 and radio-opaque marker 20 to enable a push displacement of the radio-opaque marker 20 along the radio-opaque marker delivery system 10, but also to permit easy detachment from the locating guide 14 once the anchoring members 23 and/or 24 are caught in the tumour A. It is also considered to optionally provide a stylet or any other appropriate mechanical device to assist in detaching the radio-opaque marker 20 from the locating guide 14 after penetrating the tissue, for example due to local heterogeneity in tissue composition and elasticity. Such a mechanical device could be used to exert a force onto the trailing end 21B of the radio-opaque marker 20 to release it from the locating guide 14. The assembly of FIG. 3 may come pre-assembled, such as in a sterile autoclavable pouch, in a possible non-limitative embodiment.

In a variant, the radio-opaque marker 20 is made of a monoblock piece, of medical grade material, such as gold, platinum, or even titanium, as possible metals. The material of the radio-opaque marker 20 may be radio-opaque, or detectable by any desired imaging and/or detecting modality. The radio-opaque marker 20 could have a detectable coating as a possibility.

Referring to FIGS. 4 to 6, a method for delivering a radio-opaque marker is illustrated, relative to biological tissue (e.g., tumour A). In FIG. 4, the needle 11 is in the tumour A, though it may also be adjacent to it, after a tissue sample has been obtained via the needle 11 by way of biopsy hardware (e.g., 13 in FIG. 1). In a variant, the needle 11 is not moved or minimally displaced after the tissue sample has been obtained, to remain in the target tissue. Hardware may be removed (e.g., stylet) from the proximal end of the radio-opaque marker delivery system 10, to insert the radio-opaque marker 20 and the locating guide 14 in the distal end of the tube 12, the radio-opaque marker 20 and the locating guide 14 being assembled to one another as shown in FIG. 3. In a variant, the radio-opaque marker 20 and the locating guide 14 are attached prior to insertion in the tube 12 and/or needle 11. The attachment is such that a force is required to detach the radio-opaque marker 20 from the locating guide 14, the force being greater than gravity and/or being greater than forces experience during travel through the needle 11. Various interconnection arrangements are possible between the radio-opaque marker 20 and the locating guide 14, including elastic deformation, threading engagement, or push contact.

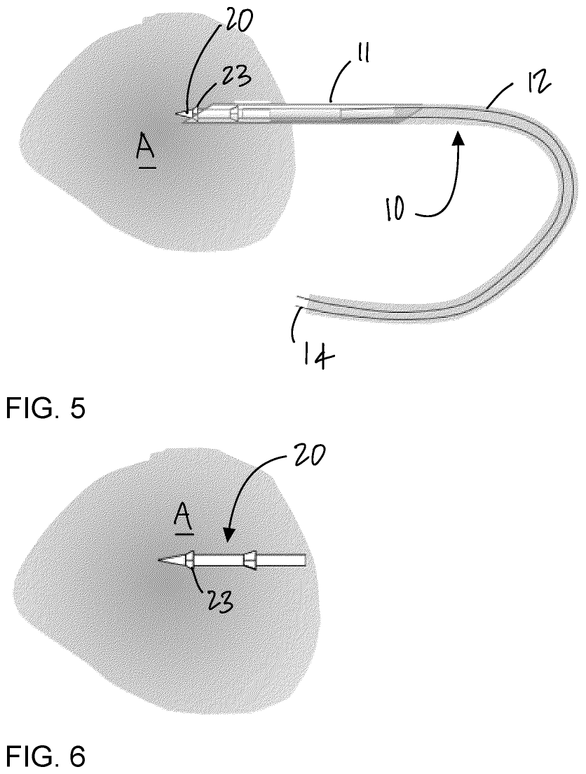

A pushing action may be exerted on the locating guide 14 for the radio-opaque marker 20 to move along the tube 12, through the needle 11, and out of the needle 11 into the tumour A. The pushing action may be extracutaneous, and may be manual or may be assisted by a pushing component. As the radio-opaque marker 20 extends out of the needle 11, the radio-opaque marker 20 penetrates the tumour A, as shown in FIG. 5. Because of the elasticity of the tumour A or like soft tissue, the penetrating tip 22 of the radio-opaque marker 20 may pierce through the tumour A or like soft tissue. A depth marker may be on the proximal end of the locating guide 14 to determine when the radio-opaque marker 20 has sufficiently penetrated the tumour A for the anchoring member(s) 23 and/or 24 to be in the soft tissue, and block any withdrawal movement of the radio-opaque marker 20. At such a point, the radio-opaque marker 20 is caught in the tumour A, and self-anchors. When the locating guide 14 is pulled out from the tube 12, the locating guide 14 detaches from the radio-opaque marker 20, as the pulling force is less than the withdrawal resistance of the radio-opaque marker 20. The withdrawal resistance may be described as the anchoring force of the radio-opaque marker 20, namely the force exerted by the soft tissue on the radio-opaque marker 20 and opposed against the marker 20 in a trailing direction.

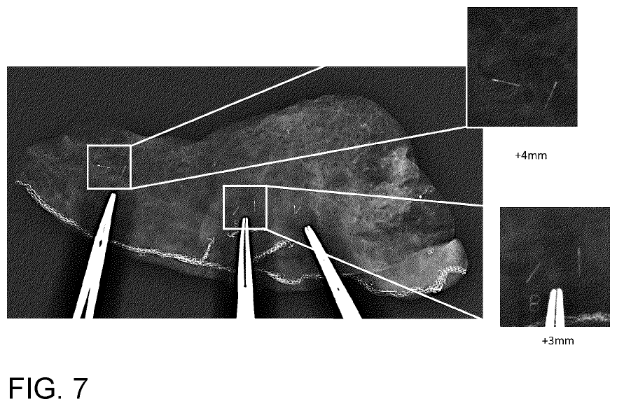

Therefore, the radio-opaque marker 20 may remain in the tumour A as shown in FIG. 6, while the needle 11 may be removed. Moreover, the step of delivering the radio-opaque marker 20 may be repeated with or without displacing the needle 11, for additional radio-opaque markers 20 to be delivered to the tumour A. Once a sufficient number of radio-opaque marker 20 has(have) been delivered to the tumour A or like soft tissue, the needle 11 may be removed.

The method illustrated in FIGS. 4 to 6 may be summarized as being for delivering a radio-opaque marker in soft tissue. The method may include positioning a marker attached to a guide inside a biopsy needle after biopsy sampling with the biopsy needle in an area of the soft tissue; with the biopsy needle remaining in the area, pushing the guide with the marker at its leading for the marker to reach the area via the needle, and pierce through the soft tissue; and pulling the guide out of the needle to detach the guide from the marker. The method may include repeating the positioning, the pushing and the pulling while the needle remains in area of the soft tissue.

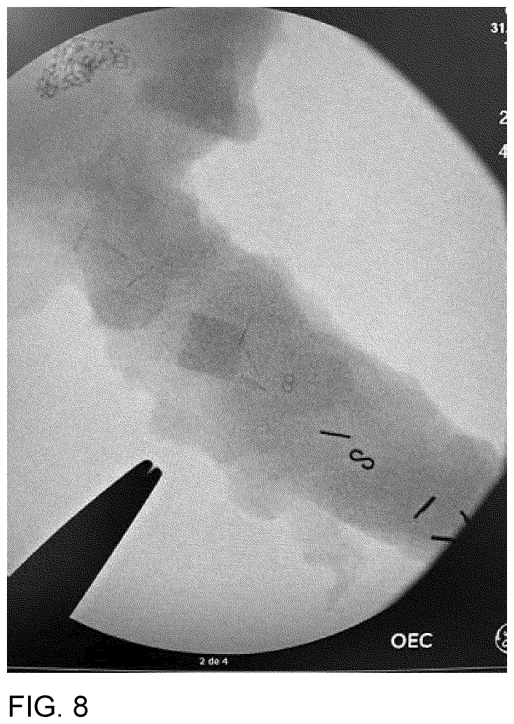

FIGS. 7 and 8 are radiographic images of a sample tissue in specific ex vivo testing, in which samples of the radio-opaque marker 20 of the present disclosure have been delivered, using the radio-opaque marker delivery system 10. The radiographic images shown the radio-opaque markers 20. In FIG. 7, there is shown an X-ray scan of a resected human liver with manually placed radio-opaque markers 20 (+2 mm, +3 mm, +4 mm). +3 mm and +4 mm versions of the radio-opaque marker 20 are highlighted in viewing windows, with a 4× zoom provided. In FIG. 8, the image shows fluoroscopic X-ray scan of a resected human oesophagus with radio-opaque markers 20 (+3 mm, +4 mm), along with other commercially available markers.

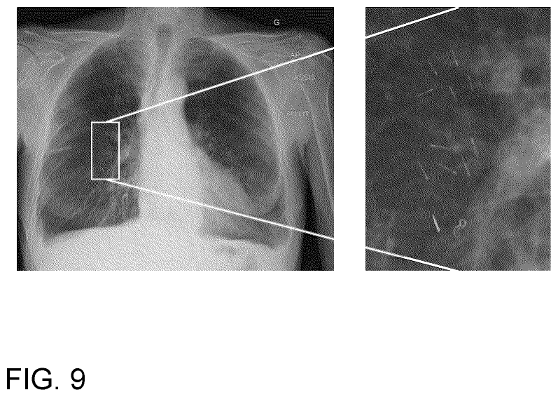

Referring to FIG. 9, a radiographic image of another embodiment of the radio-opaque marker 20 of the present disclosure is shown, positioned behind a patient as to mimic an in vivo setting. The radiographic image shows platinum-made radio-opaque markers 20 (+3 mm, +4 mm), along with other commercially available markers. +3 mm and +4 mm versions of the radio-opaque marker 20 are highlighted in the viewing window, with a 4× zoom provided. Although FIGS. 7 and 8 showed satisfactory radio-opacity of titanium-made radio-opaque markers 20, FIG. 9 illustrates that platinum-made radio-opaque markers 20 have a greater radio-contrasting visibility. The use of given materials for the radio-opaque marker 20 may allow a contrast in radiographic images. For example, with a material such as platinum, a signal-to-noise ratio (SNR) of at least 16.0 may be obtained, with FIG. 9 displaying an average SNR of 20.2. Likewise, a contrast-to-noise ratio (CNR) of at least 6.0 may be obtained, with FIG. 9 displaying an average CNR of 8.1. These values are provided only as non-restrictive examples, as some radio-opaque markers 20 in accordance with the present disclosure could have SNR and/or CNR values lower than those described above. However, if greater contrasts are desired, materials such as platinum may be used.

The radio-opaque marker delivery system 10 and radio-opaque marker 20 may simplify the marking of a tissue, as it may profit from the biopsy sampling to mark soft tissue. Thus, the overall process may be viewed as being efficient, as the use of the radio-opaque marker delivery system 10 and radio-opaque marker 20 may not require any additional invasive delivery step and/or any navigation test to locate the tumour A. The radio-opaque marker delivery system 10 and radio-opaque marker 20, such as in the case of a biopsy sampling system, may be used for tumours located in various organs, include lung, breasts, lymph nodes, prostate, liver, brain, spine, skeleton, as examples among others.

The marker 20 may be generally described as having an elongated body including a radio-opaque material, a penetrating tip tapering at a leading end of the elongated body, and one or more anchoring member(s) defining a catching surface facing toward a trailing end of the elongated body, the catching surface forming a protrusion relative a downstream portion of the elongated body.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure, and such modifications are intended to fall within the appended claims.

1. A marker comprising:
an elongated body including a radio-opaque material,
a penetrating tip tapering at a leading end of the elongated body, and
at least one anchoring member defining a catching surface facing toward a trailing end of the elongated body, the catching surface forming a protrusion relative a downstream portion of the elongated body.

2. The marker according to claim 1, wherein the marker has a circular cross-section along its entire length.

3. The marker according to claim 1, wherein the penetrating tip has a conical geometry.

4. The marker according to claim 1, wherein the at least one anchoring member has a frusto-conical body.

5. The marker according to claim 4, wherein the frusto-conical body has a cone angle greater than that of the penetrating tip.

6. The marker according to claim 4, wherein the marker has two of the anchoring member.

7. The marker according to claim 6, wherein the two anchoring members are separated by a first cylindrical segment of the elongated body.

8. The marker according to claim 7, wherein a second cylindrical segment of the elongated body is downstream of a downstream one of the two anchoring members.

9. The marker according to claim 8, wherein the first cylindrical segment has a greater diameter than the second cylindrical segment.

10. The marker according to claim 1, wherein the catching surface includes a straight radial surface.

11. The marker according to claim 1, wherein the marker is a monoblock.

12. The marker according to claim 1, wherein the marker includes platinum.

13. An assembly comprising:
a marker in accordance with claim 1; and
a guide configured to contact a trailing portion of the marker.

14. The assembly according to claim 13, wherein the guide is a silica guide.

15. The assembly according to claim 14, wherein the silica guide is a tube.

16. The assembly according to claim 13, wherein the guide is releasably attached to the trailing portion of the marker.

17. A method for delivering a radio-opaque marker in soft tissue, the method comprising:
positioning a marker and a guide inside a needle or catheter with the needle or catheter in an area of the soft tissue;
with the needle or catheter remaining in the area, pushing the guide with the marker at its leading for the marker to reach the area via the needle or catheter, and pierce through the soft tissue; and
pulling the guide out of the needle to separate the guide from the marker.

18. The method according to claim 17, further including repeating the positioning, the pushing and the pulling while the needle remains in area of the soft tissue.

19. The method according to claim 17, wherein positioning the marker attached to the guide inside the needle or catheter includes positioning the marker attached to the guide inside the biopsy needle.

20. The method according to claim 17, including positioning the needle or catheter in the area of the soft tissue to obtain at least one biopsy sample prior to positioning the marker and the guide in the area of the soft tissue.
